(19) United States
(12) Patent Application Publication
(10) Pub. No.: US 2005/0054096 A1
(43) Pub. Date: Mar. 10, 2005

(54) Pluripotent cells from monocytes, and methods of making and using pluripotent cells

(76) Inventor: Carlos J. Piniella, Miami, FL (US)

Correspondence Address: LERNER AND GREENBERG, PA, P O BOX 2480, HOLLYWOOD, FL, 33022-2480, US

(21) Appl. No.: 10/703,212

(22) Filed: Nov. 6, 2003

Related U.S. Application Data
(60) Provisional application No. 60/424,227, filed on Nov. 6, 2002.

(51) Int. Cl. C12N 5/00; C12N 5/02

(52) U.S. Cl. 435/372; 435/373

(57) ABSTRACT

Considering these findings, we claim that we have found an in-vitro culture procedure capable of conferring features of pluripotency to blood, bone marrow, and serous cavity derived mononuclear cells (serous macrophages). This procedure brings about telomerase activity in originally telomerase negative non-lymphocyte mononuclear cells. In addition, we claim that it is possible to trans-differentiate these stimulated cells into cells with hepatocellular, pancreatic, neuronal, and immunosuppressive features in vitro and in vivo.

### CROSS-REFERENCE TO RELATED APPLICATION

This application claims the benefit of U.S. Provisional Application No. 60/424,227, filed Nov. 6, 2002.

### BACKGROUND OF THE INVENTION

1. Field of the Invention

The invention relates to tissue and organ repair and replacement including replacement of the immune cells maintaining immunotolerance and suppressing autoimmunity.

2. Description of the Related Art

Monocytes are an important leukocyte subtype and are part of the mononuclear blood and bone marrow cell population. Their features are well known and extensively described in the pertinent literature; see the attached sheet of references. Various sources have suggested that monocytes despite their close relationship to granulocytic lineage do not represent generative end stage cells.

The cytokine network (CNW) mediates tissue demand. The cytokine network includes cytokines, cytokine receptors, chemokines, interleukins, growth factors, complement factors, and their receptors. Their effect may be enhanced by addition of reducing agents and alcohols.

Upon tissue demand, monocytes egress from the bone marrow or blood circulation to reappear in all tissue sites including serous cavities where they are referred to as peritoneal or pleural macrophages. Blood monocytes represent, despite their heterogeneous morphology and immunophenotype, a well-defined cell cohort. The majority actively adheres to surfaces, although a minor subpopulation may develop no adherent capabilities. The literature describes a number of techniques for exploiting adherence of these cells to separate them (“adherence technique”) and achieve high purities of over ninety-five percent (>95%). In addition to the widely used adherence technique, there are methods employing specific density (<1.077 g/mL) and centrifugation (“gradient centrifugation technique”) steps. Other techniques apply monoclonal antibodies to monocyte surface antigens like variants of CD 11, CD 14, or CD68 and couple them to fluorescent (“fluorescence-activated cell sorting”) dies or iron particles (“immunomagnetic technique”). All such methods are used to separate monocytes. Other techniques deplete the accompanying non-monocytic cells by the immunomagnetic devices. A further, less-known method utilizes elutriation pumps combined with centrifugation (“elutriation centrifugation technique”).

Monocytes can be induced to differentiate into macrophages, foreign body phagocytes, osteoklasts, antigen presenting dendritic cells, tissue mast cells, follicular dendritic cells, and brain microglia. The derivation of this array of divergent cell types has been shown in the literature.

### SUMMARY OF THE INVENTION

It is accordingly an object of the invention to provide pluripotent cells from monocytes and monocyte derived cells like those of bone marrow, blood, umbilical blood, and effusions of serous cavities (peritoneal & pleural macrophages). The object of the invention is also to provide methods of making and using pluripotent cells that overcome the hereinafore-mentioned disadvantages of the heretofore-known devices of this general type and that induce or reprogram human monocytes from blood, bone marrow, umbilical cord, and serous monocyte-derived macrophages, in vitro to develop features of pluripotency including driving monocytes into cell cycle and influencing the telomerase activity and telomere length DNA leading to enhancing proliferation activity by addition of first-step signals.

In accordance with a further object of the invention, a method for forming pluripotent monocytes includes the following steps. The first step is providing a cell population having over 90% of monocytes or serous macrophages. The next step is adding the so-called “first-step signals” to form a pluripotent monocyte population from the monocytes or serous macrophages. In addition, the method can include originating the monocytes from a source including blood, bone marrow, umbilical cord, and serous monocyte derived macrophages. The method can occur in vitro. Typically, the resulting pluripotent monocyte features reactivation of cell cycle events influencing telomerase activity and telomere DNA length and yielding enhanced proliferation activity. Monocytes not subjected to this method do no show any proliferation activity.

In accordance with a further object of the invention, the method includes trans-differentiating the pluripotent monocyte by introducing a “second-step signal”. These second-step signals enable pluripotent monocytes to mature into mesodermal, endodermal, and ectodermal somatic cells of various organs or a tissue types. The second-step signals promote and reprogram those monocytes that have been successfully subjected to the first-step signal treatment to produce every type of endodermal, mesodermal, and ectodermal somatic cells, tissues, and organs. These somatic cells also include immunocompotent cells like T- and B-lymphocytes and natural killer cells that are involved in protecting grafts against immunocompetent cells like T- and B-lymphocytes and natural killer cells that are involved in protecting grafts against immunorejections (immunotolerance) or those preventing autoimmunity. The method includes the generation of hepatocytes producing albumin, pancreatic-B-cells producing insulin, endothelial cells producing factor VIII, B-lymphocytes producing immunoglobulins, T-cells producing interleukins, cytokines, and growth factors. Likewise, the somatic cells can include B- and T-lymphocytes with rearranged immunogenes. The somatic cells can include tissue mast cells expressing tryptase, heparin, and histamine. The somatic cells also can include chondrocytes producing proteoglycanes, osteoblasts, producing osteoid, multinuclear giant cells, and endometrial cells expressing estrogen receptors and c-fms. In addition, the somatic cells can include S 100 protein producers or other nerve cells, neurons, neuroglial cells, and those producing neural products.

In accordance with a further object of the invention, initially the monocyte is telomerase negative. This contrasts the resulting pluripotent monocyte, may or may not develop a telomerase activity.

In accordance with a further object of the invention, the monocytes initially have proliferation less than one percent. This contrasts the resulting pluripotent monocyte, wherein proliferation has exceeded seventeen percent.

In accordance with a further object of the invention, a method for making first-step signals includes the following. The first step is providing an in-vitro culture of enriched monocytes from day 0 to day 7. The first step signals include the addition of one, multiple, or all of the following agents: macrophage colony stimulating factor (M-CSF), granulocyte colony stimulating factor (G-CSF), granulocyte macrophage stimulating factor (GM-CSF), interferon-gamma (INF-gamma), tumor nerosis factor-beta (INF-beta), and interleukins 1, 2, 3, 4, 5, 6, and 7 (IL 1,2,3,4,5,6,7). All of these agents are used in concentrations from five to one hundred (5-100) nanogram/mL. The culture fluid can be set with an alcohol such as methanol, ethanol, and isopropanol. Typically, the alcohol has a concentration ranging from 0.1 to 1.5 vol. %. Alcohols can be applied as vapors without directly mixing into the culture media. The method can also include setting the culture media with a reducing agent such as 2-mercaptoethanol (HSCH₂CH₂OH) and dithiotritol, in a concentration from 5 to 50 microliters per liter.

In accordance with a further object of the invention, the monocytes proliferation activity can be quantified by counting the percentage of monocytes, nuclearly binding the monoclonal antibody Mib1, Ki-S5, KiS4, or the DNA precursor 3H-thymidine. The monocytes initially bind Ki-S5 (proliferation rate) to less than one percent. In contrast, the pluripotent monocytes have a proliferation rate as measured by using Ki-S5 from 8 to 26 percent. In addition, the monocytes initially have no telomerase activity. In contrast, the resulting pluripotent monocytes may or may not have an enhanced telomerase activity of up to 199. In addition, the monocyte initially has a telomere length of 12 ∀ 7 kbp. In contrast, the pluripotent monocytes may or may not show a prolongation of their telomere length to a value of 14 ∀ 6, if needed, to match the enhanced proliferation activity.

In accordance with a further object of the invention, a method for making second-step signals includes the following steps. The initial step is providing an in-vitro culture of enriched monocytes or peritoneal macrophages that have already been treated with first-step-signals from day 0 to the day 7. The final step is treating the pluripotent monocytes with a tissue-specific environmental factor. The tissue-specific environmental factor can be any one of the following:
1. a cell-free tissue extract,
2. an organ extract,
3. a co-culture (co-incubation) of the pluripotent monocytes with suspended viable cells of the target tissues, cell group, or organ, and
4. inoculation of the pluripotent monocytes into the organs that are in need of replacement or repair.

The tissue-specific environmental factor is preferably added between day 6 and day 15 and is added in vitro.

An additional step includes injecting the monocytes after the first and/or second-step signal treatment into the artery supplying the target organ to be treated or into the solid tissue directly when it need repair or substitution.

The invention also encompasses a mononuclear blood cell. The mononuclear blood cell can have a surface expression of CD45, CD11, CD14, and CD68. The mononuclear blood cell is potentially phagocytic and shows active phagocytosis when set with particulate matter. The mononuclear blood cell contains lysosomal acid esterase detected by the substrate alpha naphthyl acetate as a serin-esterase with the well-known specific isoenzymes with the main band containing over 70% of total enzyme activity. The mononuclear blood cell can have oncogen-product c-fms having a monocyte-specific methylation pattern in a first exon of its promoter region. The mononuclear blood cell preferably has negligible or no telomerase activity and a Ki-S5-measured proliferation activity less than one percent.

In accordance with a further object of the invention, a method for reprogramming mononuclear blood cells includes the following steps. The first step is separating and culturing in vitro using culture media. The media can include RPMI, 2 to 20% fetal calf sera, 2 to 20% of adult human sera, sera prepared from human umbilical cord, human ABO sera. The culturing can be maintained in vitro from day 0 to day 14. An additional step can be, from day 0, supplementing the in vitro culture with 5-20% FCS and a first-step signal. Possible first-step signals include a macrophage colony stimulating factor (MCSF) at concentrations of 5 to 100 nanogram per mL, granulocyte colonies stimulating factor (G-CSF) at a concentration of 5 to 100 nanogram per mL, interleukin-1, 2, 3, 4, 5, 6, and 7 (IL-1,2,3,4,5,6, and7) at concentrations of 5 to 80 nanogram per ml, interferon gamma (INF-g) at concentrations of 1 to 80 nanogram per mL, stem cell factor (SCF) at concentrations of 5 to 100 nanogram per mL, tumor necrosis factor beta (TNF-beta) at concentrations of 5 to 80 nanogram per mL, and leukemia inhibitory factor (LIF) at concentrations of 5 to 30 nanogram per mL. In addition, cortical steroids such as metadextrone can be added at concentrations of 10-100 microgram per milliliter.

In accordance with a further object of the invention, a method for confirming proliferation activity includes measuring telomerase activity daily.

In accordance with a further object of the invention, a cultured cell from monocytes or monocyte-derived cells produce specific proteins. The proteins include cell surface proteins (membrane proteins), cytoplasmic proteins, or nuclear proteins. For example, the protein could be CD178 (Fas-Ligand), CD 90 (FY-1), CD123 (interleukine-3 receptor alpha), CD135 (Growth Factor Receptor), or CD 117 (c-kit or stem cell factor receptor).

In accordance with a further object of the invention, a pluripotent cell can be used for trans-differentiation into many different cell types, developing phenotypes, functions, and morphology of nearly all other human somatic cells of mesodermal, ectodermal, and endodermal origin.

In accordance with a further object of the invention, a method for trans-differentiating a pluripotent cell, also referred to as an adult stem cell, generated from a monocyte or monocyte-derived cell includes the following steps. Under the influence of the first-step signals, monocytes or monocyte-derived cells enter the cell cycle and acquire enhanced proliferation capabilities, during which telomerase activity may or may not be enhanced. This step includes maintaining of monocytes and monocyte derived cells in a culture media from day 0 to day 7 under the influence of the first-step signals detailed above in order to achieve pluripotent adult stem cells. The next step is trans-differentiating the pluripotent cells in vivo or in vitro. Under in vitro conditions, the pluripotent adult stem cells kept in culture media are supplemented with the so-called second-step signals from day 6 or 7 on. In this step, pluripotent cells derived from monocytes trans-differentiate into terminally differentiated human organ specific cell types.

In accordance with a further object of the invention, a method for manufacturing second-step signals includes the following steps. The culture media containing pluripotent monocytes are set with alcohols such as methanol, ethanol, or isopropanol in minor concentrations of 0.01 vol. % or exposed to alcohol vapor alone or in various combinations with and without addition of reducing agents such as 2-mercaptoethanol (HSCH₂CH₂OH) dithiotritol in concentrations of 5 to 40 micrometers per liter culture medium alone or in various combinations and final molarity alone or in various combination with retinoic acid, forbolic acid ester, and vitamin D3 in concentrations of 1 to 80 nanogram per milliliter. Preferably, the alcohols(methanol, ethanol, or isopropanol in concentrations from 0.1 to 1.5 vol % are added to the culture media. In some instances, exposing the culture to an alcohol vapor has been sufficient. In addition, the culture medium can be set with a reducing agent including 2-mercaptoethanol (HSCH₂CH₂OH) and dithiotritol. Preferably, the reducing agent has a concentration from 5 to 50 microliter per liter of the culture medium. In addition, interleukin 2, 3, 5, and 7 alone or in combination with a cytokine, a chemokine, an interleukin, a growth factor, and a complement factor can be used to set the culture medium. Examples of complement factors include a stem cell factor (SCF), a leukemia inhibitory factor (LIV), and a growth Factor (GF). A possible additional step is waiting from five to seven days, and incubating the culture cells with a cell free S100-supernatant of fresh sonicated human tissue types or organs needing repair or substitution for two to four further days. The fresh sonication-lysed human tissue type or organs can be skin, lymph node, pancreas, liver, bone marrow, brain, major nerves, endothelia, blood cells, or muscular tissue.

In accordance with a further object of the invention, a method for detecting monocytes incubated with live extract includes detecting a liver cell protein with specific monoclonal antibodies. Examples of liver proteins include cytokeratin and albumin, or other well known enzymes specifically produced in the liver for certain metabolic reactions.

In accordance with a further object of the invention, a method for detecting monocytes incubated with lymph-node extract includes detecting cytotoxic and natural killer cell activity; and detecting a suppression of in-vitro cytotoxicity and detecting CD178 positivity in these cells.

In accordance with a further object of the invention, a method for detecting monocytes or peritoneal macrophages incubated with brain extract includes detecting at least one of the antigens such as S100 and neuron specific enolase.

In accordance with a further object of the invention, a method for repairing tissue or an organ includes applying in vivo pluripotent monocytes into the tissue or the organs. More specifically, pluripotent cells produced as detailed above can be applied to a pancreatic artery or direct injection into the solid gland tissue of a diabetic patient. Then, the monocyte derived pluripotent cells can terminally differentiate to form pancreatic island B-cells capable of producing insulin. The pluripotent cells derived from monocytes in vitro can be applied in vivo to a diseased liver via a portal vein and terminally differentiated to form hepatocytes. The monocyte-derived pluripotent cells can be applied to an injured nerve to terminally differentiate into neural cells. The pluripotent monocyte can be applied into a neighborhood of an infarcted heart area to terminally differentiate into cardial myocytes.

In such procedures, the monocytes are at a concentration of 1 to 5×10⁷.

In accordance with a further object of the invention, a method for in vitro induction of cell cycle activity and proliferation in human adherent mononuclear cells rich in monocytes and macrophages with the immunophenotype and other features detailed above is included. In this step, telomerase activity and telomere length may or may not increase.

In accordance with a further object of the invention, the invention encompasses a method for in vivo induction of pluripotency including the corresponding immunophenotype in human adherent mononuclear cells rich in monocytes or macrophages with the immunophenotype detailed above.

In accordance with a further object, the invention encompasses a method for in vitro induction of cells produced or modified into terminally trans-differentiated organ-specific cells exemplified by pancreatic island B-cells, hepatocytes, nerve or neural cells, lymphoid cells, brain cells, cardiac myocytes, and endothelial cells. The method is capable of suppression of auto- and allergenic immune reaction otherwise leading to graft rejection or the well-known list of autoimmune diseases like primary chronic polyarthritis (PCP) and other rheumatic diseases.

Other features that are considered as characteristic for the invention are set forth in the appended claims.

Although the invention is illustrated and described herein as embodied in pluripotent cells from monocytes, and methods of making and using pluripotent cells, it is, nevertheless, not intended to be limited to the details shown since various modifications and structural changes may be made therein without departing from the spirit of the invention and within the scope and range of equivalents of the claims.

The construction and method of operation of the invention, however, together with additional objects and advantages thereof, will be best understood from the following description of specific embodiments when read in connection with the accompanying examples.

### DETAILED DESCRIPTION OF PREFERRED EMBODIMENTS

The biological mechanism of this two-step trans-differentiation process is described below.

In the first step, non-lymphocytic mononuclear human blood cells are converted to pluripotent, adult stem cell like cells. This step is completed in vitro, in a culture. The change results from exposure to the first-step factors (first-step signals).

In the second step, pluripotent, stem-cell-like cells are converted to organ specific cells. This conversion (trans-differentiation) is either completed in-vitro by adding to the cell cultures of the pluripotent cells, the second-step factors (second-step signals) and the subsequent injection of the transdifferentiated cells into tissue or organs wanting of repair or substitution. The final subcellular changes of these cells on a molecular level during the describe process remains to be cleared by gene array and proteomic studies.

An alternative to the proposed methods provides for the trans-differentiation of pluripotent monocytes, the cells are exposed to the second-step signals including cell-free S100 supernatant prepared from homogenized or lysed fresh organs after centrifugation at 100,000 G for 30 minutes. Alternatively, organ cells or enriched cell populations such as lymphocytes can be utilized as feeder layers and co-cultures and washed away before reinjecting of such monocyte derived cells for the treatment.

1. A method for forming pluripotent monocytes, which comprises:
providing a monocyte; and
adding a first-step signal to form a pluripotent monocyte from the monocyte.

2. The method according to claim 1, which further comprises originating the monocyte from a source selected from the group consisting of blood, bone marrow, umbilical cord, and serous monocyte-derived macrophages.

3. The method according to claim 1, wherein the adding step occurs in vitro.

4. The method according to claim 1, wherein the pluripotent monocyte has a feature selected from the group consisting of reactivation of telomerase, elongation of telomere DNA, and enhanced proliferation activity.

5. The method according to claim 1, which further comprises trans-differentiating the pluripotent monocyte by introducing a second-step signal to form mesodermal, endodermal, and ectodermal somatic cells of at least one of an organ and a tissue type.

6. The method according to claim 5, wherein the second-step signal is introduced to all mosodermal, endodermal, and ectodermal somatic cells of the at least one of an organ and a tissue type.

7. The method according to claim 5, wherein the second-step signal is introduced to all mesodermal, endodermal, and ectodermal somatic cells of every and every tissue type.

8. The method according to claim 5, wherein the somatic cells include natural killer cells protecting grafts.

9. The method according to claim 5, wherein the somatic cells include hepatocytes producing albumin.

10. The method according to claim 5, wherein the somatic cells include pancreatic-B cells producing insulin.

11. The method according to claim 5, wherein the somatic cells include endothelium producing factor VIII.

12. The method according to claim 5, wherein the somatic cells include all features and products of endothelial cells.

13. The method according to claim 5, wherein the somatic cells include B- and T-lymphocytes with rearranged immunogenes.

14. The method according to claim 5, wherein the somatic cells include tissue mast cells expressing tryptase, heparin, and histamine.

15. The method according to claim 5, wherein the somatic cells include chondrocytes producing proteoglycanes.

16. The method according to claim 5, wherein the somatic cells include osteoblasts.

17. The method according to claim 5, wherein the somatic cells include multinuclear giant cells.

18. The method according to claim 5, wherein the somatic cells include endometrial cells expressing estrogen receptor and c-fins.

19. The method according to claim 5, wherein the somatic cells include S100 protein producers selected from the group consisting of nerve cells, neurons, neuroglial cells, and neural products.

20. The method according to claim 1, wherein the monocyte is telomerase negative.

21. The method according to claim 1, wherein the pluripotent monocyte is telomerase positive.

22. The method according to claim 1, wherein the monocyte has proliferation less than one percent.

23. The method according to claim 1, wherein the pluripotent monocyte has proliferation exceeding seventeen percent.

24. A method for making first-step signals, which comprises:
providing an in-vitro culture of enriched monocytes from day 0 to day 7;
adding macrophage colony stimulating factor (M-CSF), granulocyte colony stimulating factor (G-CSF), granulocyte macrophage stimulating factor (GM-CSF), interferon-gamma (INF-gamma), tumor nerosis factor-beta (INF-beta), and interleukins 2,3,5, and 7 (IL2,3,5,7), all in concentrations of 5-80 nanogram/mL.

25. The method according to claim 24, which further comprises setting the cultures with an alcohol.

26. The method according to claim 25, wherein the alcohol is selected from the group consisting of methanol, ethanol, and isopropanol.

27. The method according to claim 25, wherein the alcohol has a concentration ranging from 0.1 to 1.5 vol. %.

28. The method according to claim 24, which further comprises setting the culture media with a reducing agent.

29. The method according to claim 28, wherein the reducing agent is selected from the group consisting of 2-mercaptoethanol (HSCH₂CH₂OH) and dithiotritol.

30. The method according to claim 28, wherein the reducing agent has a concentrations from 5 to 50 microliters per Liter.

31. The method according to claim 1, wherein the monocyte has a proliferation rate Ki-S5 of less than one percent.

32. The method according to claim 1, wherein the pluripotent monocyte has proliferation rate Ki-S5 from 8 to 26 percent.

33. The method according to claim 1, wherein the monocyte has a telomerase activity from 4 to 12.

34. The method according to claim 1, wherein the pluripotent monocyte has a telomerase activity of 199.

35. The method according to claim 1, wherein the monocyte has a telomere length from 5 to 19 kbp.

36. The method according to claim 35, wherein the pluripotent monocyte has a telomere length from 9 to 19.

37. A method for making second-step signals, which comprises:
providing an in-vitro culture of enriched monocytes after treatment with first-step-signals from day 0 to the day 7; and
subsequently treating the culture with a tissue-specific environmental factor.

38. The method according to claim 37, wherein the tissue-specific environmental factor is a tissue extract.

39. The method according to claim 37, wherein the tissue-specific environmental factor is an organ extract.

40. The method according to claim 37, wherein the tissue-specific environmental factor is added from day 7 to day 14.

41. The method according to claim 37, wherein the tissue-specific environmental factor is added in vitro.

42. The method according to claim 37, which further comprises injecting the stimulated monocytes from day 4 to 7 into an artery supplying the organ to be treated.

43. The method according to claim 37, which further comprises directly injecting the stimulated monocytes from day 4 to 7 into solid tissue needing repair or substitution.

44. A mononuclear blood cell.

45. The mononuclear blood cell according to claim 44, wherein said mononuclear blood cell has a surface expression of CD45, CD11, CD14, CD68.

46. The mononuclear blood cell according to claim 44, wherein said mononuclear blood cell has potentially phagocytic and show active phagocytoses when set with particulate matter.

47. The mononuclear blood cell according to claim 44, wherein said mononuclear blood cell contains lysosomal acid esterase detected by the substrate alpha naphthyl acetate as a serin-esterase with the well known specific isoenzymes with the main band containing over 70% of total enzyme activity.

48. The mononuclear blood cell according to claim 44, wherein said mononuclear blood cell has oncogen-product c-fins having a monocyte-specific methylation pattern in a first exon of its promoter region.

49. The mononuclear blood cell according to claim 44, wherein said mononuclear blood cell has no telomerase activity.

50. The mononuclear blood cell according to claim 44, wherein said mononuclear blood cell has negligible telomerase activity.

51. The mononuclear blood cell according to claim 44, wherein said mononuclear blood cell has a Ki-S5-measured proliferation activity less than one percent.

52. A method for making mononuclear blood cells, which comprises:
separating and culturing in vitro using media.

53. The method according to claim 52, wherein the media includes RPNO.

54. The method according to claim 52, wherein the media contains from 2 to 20% fetal calf sera.

55. The method according to claim 52, wherein the media contains from 2 to 20% of adult human sera.

56. The method according to claim 52, wherein the media contains sera prepared from human umbilical cord.

57. The method according to claim 52, wherein the media contains ABO blood.

58. The method according to claim 52, which further comprises culturing in vitro from day 0 to day 14.

59. The method according to claim 52, which further comprises, from day 0, supplementing the in vitro culture with 5-20% FCS and a first-step signal.

60. The method according to claim 59, wherein the first-step signal is selected from the group consisting of a macrophage colony stimulating factor (M-CSF) at concentrations of 5 to 80 nanogram per mL of culture fluid, granulocytes-macrophage colony stimulating factor (GM-CSF) at a concentration of 5 to 80 nanogram per mL, granulocyte colony stimulating factor (G-CSF) at a concentration of 5 to 80 nanogram per mL, Interleukin-2, 3, 5 and 7 (IL-2,3,5,7) at concentrations of 5 to 80 nanogram per ml, interferon gamma (INF-g) at concentrations of 1 to 80 nanogram per mL, stem cell factor (SCF) at concentrations of 5 to 80 nanogram per mL, tumor necrosis factor beta (TNF-beta) at concentrations of 5 to 80 nanograin per mL, and Leukemia inhibitory factor (LIF) at concentrations of 5 to 30 nanogram per mL.

61. A method for confirming proliferation activity, which comprises:
measuring telomerase activity daily; and
checking the telomerase activity for a sudden rise.

62. A cultured cell from a monocyte or a monocyte-derived cell, comprising a protein, said protein being selected from the group consisting of a cell-surface-membrane protein and a cytoplasmic protein.

63. The cultured cell according to claim 62, wherein said protein is CD178 (Fas-Ligand).

64. The cultured cell according to claim 62, wherein said protein is CD 90 (Thyl).

65. The cultured cell according to claim 62, wherein said protein is CD123 (Interleukine-3-Receptor-alpha).

66. The cultured cell according to claim 62, wherein said protein is CD1 3 5 (Growth Factor Receptor).

67. The cultured cell according to claim 62, wherein said protein is CD 117 (c-kit or Stem Cell factor Receptor).

68. A pluripotent cell for trans-differentiation into many different cell types, developing phenotypes, function, and morphology of nearly all other human cells of mesodermal, ectoderm, and endodermal origin.

69. A method for trans-differentiating a pluripotent cell generated from a monocyte or monocyte-derived cell, which comprises:
acquiring high telomerase activity;
maintaining a culture media through new cell cycles from day 0 to day 7; and
trans-differentiating the pluripotent cell by supplementing the pluripotent cell with a second-step signal between day 0 to 7 into terminally-differentiated, human, organ-specific cell types.

70. A method for manufacturing second-step signals, which comprises:
setting a culture media with an alcohol;
setting the culture media with M-CSF and GM-CSF; and
adding retinoic acid, phorbolic acid ester, and vitamin D3 when cell proliferation is low, all in concentrations of 1-80 nanogram per ml.

71. The method according to claim 70, wherein the alcohol is selected from the group consisting of methanol, ethanol, and isopropanol.

72. The method according to claim 70, wherein the alcohol has a concentrations from 0.1 to 1.5 vol. %.

73. The method according to claim 70, wherein the alcohol is a vapor.

74. The method according to claim 70, which further comprises setting the culture medium with a reducing agent.

75. The method according got claim 74, wherein the reducing ageing is selected from the group consisting of 2-mercaptoethanol (HSCH₂CH₂OH) and dithiotritol.

76. The method according to claim 74, wherein the reducing agent has a concentration from 5 to 50 microliter per liter of the culture medium.

77. The method according to claim 70, which further comprises setting the culture media with at least one interleukin [2, 3, 5, and 7 alone or in combination] with a cytokine, a chemokine, an interleukin, a growth factor, and a complement factor.

78. The method according to claim 77, wherein the at least one interlukin is selected from the group consisting of interleukin 2, interleukin 3, interleukin 5, and interleukin 7.

79. The method according to claim 77, wherein the complement factor is selected from the group consisting of a stem cell factor (SCF), a leukemia inhibitory factor (LIF), and growth Factor (GF).

80. The method according to claim 70, which further comprises:
waiting from five to seven days; and
incubating the culture cells with a cell free S 100 supernatant of fresh sonication-lysed human tissue types or organs needing repair or substitution for two to four further days.

81. The method according to claim 80, wherein the fresh sonication-lysed human tissue type or organs are selected from the group consisting of skin, lymph node, pancreas, liver, bone marrow, and brain.

82. A method for detecting monocytes incubated in pancreatic extract, which comprises detecting a pancreatic protein with corresponding specific antibodies.

83. The method according to claim 82, wherein the pancreatic protein is selected from the group consisting of a cytoplasmic protein, cytokeratin, glycagon, and insulin.

84. A method for detecting monocytes incubated with liver extract, which comprises detecting a liver protein with specific monoclonal antibodies by immunocytochemistry.

85. The method according to claim 84, wherein the liver protein is selected from the group consisting of cytokeratin and albumin.

86. A method for detecting monocytes incubated with lymph-node extract, which comprises:
detecting cytotoxic and natural killer cell activity;
detecting a suppression of in-vitro cytotoxicity; and
detecting positive CD 178.

87. A method for detecting monocytes incubated with brain extract, which comprises detecting at least one of antigen S 100 and neuron specific enolase.

88. A method for repairing tissue or an organ, which comprises applying in-vivo monocytes cells to the tissue or the organ.

89. The method according to claim 88, which further comprises:
applying the monocyte to a diabetic pancreas via pancreatic artery; and
terminally differentiating the monocyte to form an island B cell.

90. The method according to claim 88, which further comprises:
applying the monocyte to a diseased liver via a hepatic vein; and
terminally differentiating the monocyte to form a hepatocyte.

91. The method according to claim 88, which further comprises:
applying the monocyte to an injured nerve; and
terminally differentiating the monocyte to form a nerve cell.

92. The method according to claim 88, which further comprises:
applying the monocyte to an infarcted heart area; and
terminally differentiating the monocyte to form a cardial myocyte.

93. The method according to claim 88, wherein the monocytes are at a concentration of 1 to 5×10⁷.

94. A method for in-vitro induction of telomerase activity, telomere elongation and enhanced proliferation activity in human adherent mononuclear cells rich in monocytes or macrophages with the immunophenotype detailed above.

95. A method for in-vitro induction of pluripotency including the corresponding immunophenotype in human adherent mononuclear cells rich in monocytes or macrophages with the immunophenotype detailed above.

96. A method for in-vitro induction of cells into terminally trans-differentiated organ-specific cells exemplified by pancreatic island cells, hepatocytes, nerve or neural cells, lymphoid cells capable of suppression of auto- and allogenic immune reaction otherwise leading to graft rejection or the well known list of auto-immune diseases like primary chronic polyarthritis (PCP).
